SEQUENCE LISTING (24 sequences)

SEQ ID NO: 1 - DNA, 7128 bp, synthetic construct
  source 1..7128: mol_type = other DNA
tgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcttctgtagggcgcagtagtccagggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagaccaccatgggcgtgatcaagcccgacatgaagatcaagctgaagatggagggcaacgtgaacggccacgccttcgtgatcgagggcgagggcgagggcaagccctacgatggcaccaacaccatcaacctggaggtgaaggagggcgcccccctgcccttcagctacgacatcctgaccaccgccttcagctacggcaacagggccttcaccaagtaccctgacgacatccccaactacttcaagcagagcttccccgagggctacagctgggagaggaccatgaccttcgaggacaagggcatcgtgaaggtgaagagcgacatcagcatggaggaggacagcttcatctacgagatccacctgaagggcgagaacttcccccccaacggccccgtgatgcagaaggagaccaccggctgggacgccagcaccgagaggatgtacgtgagggacggggtgctgaagggggacgtgaagatgaagctgctgctggagggcggcggccaccacagggtggacttcaagaccatctacagggccaagaaggccgtgaagctgcccgactaccacttcgtggaccacaggatcgagatcctgaaccacgacaaggactacaacaaggtgaccgtgtacgagatcgcagtggccaggaacagcaccgatggcatggacgagctgtacaaggacatctccaagtttgcagagaaagacaatatagttcttggagaaggtggaatcacactgagtggaggtcaatgagcaagaatttctttagcaggtagcggaggaggaggtcccaagaagaagaggaaggtcgaccccaagaagaagaggaaggtcgaccccaagaagaagaggaaggtcagggtgagtctatgggacgcttgatgttttctttccccttcgagtccaagctaggcccttttgctaatcatgttcatacctcttatcttcctcccacagctccagccaccatggagcggaggaggagtgagcaagggcgaggccgtgatcaaggagttcatgaggttcaaggtgcacatggagggcagcatgaacggccacgagttcgagatcgagggcgagggcgagggcaggccctacgagggcacccagaccgccaagctgaaggtgaccaagggcggccccctgcccttcagctgggacatcctgagcccccagttcatgtacggcagcagggccttcaccaagcaccccgccgacatccccgactactacaagcagagcttccccgagggcttcaagtgggagagggtgatgaacttcgaggacggcggcgccgtgaccgtgacccaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggaggacgccaagaacatcaagaagggccccgcccccttctaccccctggaggatggcaccgcaggcgagcagctgcacaaggccatgaagaggtacgccctggtgcccggggccatcgccttcaccgacgcccacatccaggtggacgtgacctacgcagagtacttcgagatgagcgtgaggctggccgaggccatgaggaggtacggcctgaacaccaaccacaggatcgtggtgtgcagcgagaacagcagccagttcttcatgcccgtgctgggcgccctgttcatcggggtggccgtggcccccgccaacgacatctacaacgagagggagctgctgaacagcatgggcatcagccagcccaccgtggtgttcgtgagcaagaagggcctgaggaaggtgctgaacgtgcagaagaagctgcccatcatcaggaagatcatcatcatggacagcaagaccgactaccagggcttccagagcatgtacaccttcgtgaccagccacctgccccccagcttcaacgagtatgacttcgtgcccgagagcttcgacagggacaagaccatcgccctgatcatgaacagcagcggcagcaccggcctgcccaagggcgtggccctgccccacaggaccgcctgcgtgaggttcagccacgccagggaccccatcttcggctaccagaacatccccgacaccgccatcctgagcgtggtgcccttccaccacggcttcggcatgttcaccaccctgggctacctgatctgcggcttcagggtggtgctgatgtacaggttcgaggaggagctgttcctgaggagcctgcaggactacaagatccagagcgccctgctggtgcccaccctgttcagctgcctggccaagagcaccctgatcgacaagtacgacctgagcagcctgagggagatcgcttcaggtggcgcccccctgagcaaggaggtgggcgaggccgtggccaagaggttcaggctgcccggcatcaggcagggctacggcctgaccgagaccaccaacgccgtgatgatcacccccgagggcgacaggaagcccggcagcgtgggcaaggtggtgcccttcttcgaggccaaggtggtggacctggtgaccggcaagaccctgggcgtgaaccagaggggcgagctgtgcgtgaggggccccatgatcatgagcggctacgtgaacaaccccgaggccaccaacgccctgatcgacaaggacggctggctgcacagcggcgacatcgcctactgggacgaggacgagcacttcttcatcgtggacaggctgaagagcctgatcaagtacaagggctaccaggtggcccccgccgagctggagggcatcctgctgcagcacccctacatctttgacgcaggcgtggccggcctgccagatgacgacgcaggagagctgcccgcagctgtggtggtgctggagcacggcaagaccatgaccgagaaggagatcgtggactacgtggccagccaggtgaccaccgccaagaagctgaggggaggcgtggtgttcgtggacgaggtgcccaggggcagcaccggcaagctggacgccaggaagatcagggagatcctgaccaaggccaagaaggacggcaagatcgccgtgtaacgctttcttgctgtccaatttctattaaaggttcctttgttccctaagtccaactactaaactgggggatattatgaagggccttgagcatctggattctgcctacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagct

SEQ ID NO: 2 - DNA, 87 bp, synthetic construct
  source 1..87: mol_type = other DNA
ggtagcggaggaggaggtcccaagaagaagaggaaggtcgaccccaagaagaagaggaaggtcgaccccaagaagaagaggaaggtc

SEQ ID NO: 3 - DNA, 87 bp, synthetic construct
  source 1..87: mol_type = other DNA
ggtagcggaggaggaggtcccaagaagaagaggaaagttgatccaaagaagaagaggaaagttgacccaaagaagaagaggaaggtc

SEQ ID NO: 4 - DNA, 81 bp, synthetic construct
  source 1..81: mol_type = other DNA
tacccctacgacgtgcccgactacgcctacccctacgacgtgcccgactacgcctacccctacgacgtgcccgactacgcc

SEQ ID NO: 5 - DNA, 102 bp, Homo sapiens
  source 1..102: mol_type = other DNA
agggtgagtctatgggacgcttgatgttttctttccccttcgagtccaagctaggcccttttgctaatcatgttcatacctcttatcttcctcccacagctc

SEQ ID NO: 6 - DNA, 139 bp, Homo sapiens
  source 1..139: mol_type = other DNA
ggcaggttggtatcaaggttacaagacaggtttaaggagaccaatagaaactgggcatgtggagacagagaagactcttgggtttctgataggcactgactctctctgcctattggtctattttcccacccttaggctg

SEQ ID NO: 7 - DNA, 24 bp, synthetic construct
  source 1..24: mol_type = other DNA
cagccaccatggagcggaggagga

SEQ ID NO: 8 - DNA, 36 bp, synthetic construct
  source 1..36: mol_type = other DNA
cagccaccatggtcaagatccacgagcggaggagga

SEQ ID NO: 9 - DNA, 27 bp, synthetic construct
  source 1..27: mol_type = other DNA
cagaaccaccatggagccggaggagga

SEQ ID NO: 10 - DNA, 7128 bp, synthetic construct
  source 1..7128: mol_type = other DNA
tgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcttctgtagggcgcagtagtccagggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagaccaccatgggcgtgatcaagcccgacatgaagatcaagctgaagatggagggcaacgtgaacggccacgccttcgtgatcgagggcgagggcgagggcaagccctacgatggcaccaacaccatcaacctggaggtgaaggagggcgcccccctgcccttcagctacgacatcctgaccaccgccttcagctacggcaacagggccttcaccaagtaccctgacgacatccccaactacttcaagcagagcttccccgagggctacagctgggagaggaccatgaccttcgaggacaagggcatcgtgaaggtgaagagcgacatcagcatggaggaggacagcttcatctacgagatccacctgaagggcgagaacttcccccccaacggccccgtgatgcagaaggagaccaccggctgggacgccagcaccgagaggatgtacgtgagggacggggtgctgaagggggacgtgaagatgaagctgctgctggagggcggcggccaccacagggtggacttcaagaccatctacagggccaagaaggccgtgaagctgcccgactaccacttcgtggaccacaggatcgagatcctgaaccacgacaaggactacaacaaggtgaccgtgtacgagatcgcagtggccaggaacagcaccgatggcatggacgagctgtacaaggacatctccaagtttgcagagaaagacaatatagttcttggagaaggtggaatcacactgagtggaggtcaacgagcaagaatttctttagcaggtagcggaggaggaggtcccaagaagaagaggaaggtcgaccccaagaagaagaggaaggtcgaccccaagaagaagaggaaggtcagggtgagtctatgggacgcttgatgttttctttccccttcgagtccaagctaggcccttttgctaatcatgttcatacctcttatcttcctcccacagctccagccaccatggagcggaggaggagtgagcaagggcgaggccgtgatcaaggagttcatgaggttcaaggtgcacatggagggcagcatgaacggccacgagttcgagatcgagggcgagggcgagggcaggccctacgagggcacccagaccgccaagctgaaggtgaccaagggcggccccctgcccttcagctgggacatcctgagcccccagttcatgtacggcagcagggccttcaccaagcaccccgccgacatccccgactactacaagcagagcttccccgagggcttcaagtgggagagggtgatgaacttcgaggacggcggcgccgtgaccgtgacccaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggaggacgccaagaacatcaagaagggccccgcccccttctaccccctggaggatggcaccgcaggcgagcagctgcacaaggccatgaagaggtacgccctggtgcccggggccatcgccttcaccgacgcccacatccaggtggacgtgacctacgcagagtacttcgagatgagcgtgaggctggccgaggccatgaggaggtacggcctgaacaccaaccacaggatcgtggtgtgcagcgagaacagcagccagttcttcatgcccgtgctgggcgccctgttcatcggggtggccgtggcccccgccaacgacatctacaacgagagggagctgctgaacagcatgggcatcagccagcccaccgtggtgttcgtgagcaagaagggcctgaggaaggtgctgaacgtgcagaagaagctgcccatcatcaggaagatcatcatcatggacagcaagaccgactaccagggcttccagagcatgtacaccttcgtgaccagccacctgccccccagcttcaacgagtatgacttcgtgcccgagagcttcgacagggacaagaccatcgccctgatcatgaacagcagcggcagcaccggcctgcccaagggcgtggccctgccccacaggaccgcctgcgtgaggttcagccacgccagggaccccatcttcggctaccagaacatccccgacaccgccatcctgagcgtggtgcccttccaccacggcttcggcatgttcaccaccctgggctacctgatctgcggcttcagggtggtgctgatgtacaggttcgaggaggagctgttcctgaggagcctgcaggactacaagatccagagcgccctgctggtgcccaccctgttcagctgcctggccaagagcaccctgatcgacaagtacgacctgagcagcctgagggagatcgcttcaggtggcgcccccctgagcaaggaggtgggcgaggccgtggccaagaggttcaggctgcccggcatcaggcagggctacggcctgaccgagaccaccaacgccgtgatgatcacccccgagggcgacaggaagcccggcagcgtgggcaaggtggtgcccttcttcgaggccaaggtggtggacctggtgaccggcaagaccctgggcgtgaaccagaggggcgagctgtgcgtgaggggccccatgatcatgagcggctacgtgaacaaccccgaggccaccaacgccctgatcgacaaggacggctggctgcacagcggcgacatcgcctactgggacgaggacgagcacttcttcatcgtggacaggctgaagagcctgatcaagtacaagggctaccaggtggcccccgccgagctggagggcatcctgctgcagcacccctacatctttgacgcaggcgtggccggcctgccagatgacgacgcaggagagctgcccgcagctgtggtggtgctggagcacggcaagaccatgaccgagaaggagatcgtggactacgtggccagccaggtgaccaccgccaagaagctgaggggaggcgtggtgttcgtggacgaggtgcccaggggcagcaccggcaagctggacgccaggaagatcagggagatcctgaccaaggccaagaaggacggcaagatcgccgtgtaacgctttcttgctgtccaatttctattaaaggttcctttgttccctaagtccaactactaaactgggggatattatgaagggccttgagcatctggattctgcctacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagct

SEQ ID NO: 11 - DNA, 13 bp, synthetic construct
  source 1..13: mol_type = other DNA
ataacttcgtata

SEQ ID NO: 12 - DNA, 13 bp, synthetic construct
  source 1..13: mol_type = other DNA
taccgttcgtata

SEQ ID NO: 13 - DNA, 13 bp, synthetic construct
  source 1..13: mol_type = other DNA
tatacgaagttat

SEQ ID NO: 14 - DNA, 32 bp, synthetic construct
  source 1..32: mol_type = other DNA
taactttaaataatgccaattatttaaagtta

SEQ ID NO: 15 - DNA, 32 bp, synthetic construct
  source 1..32: mol_type = other DNA
taactttaaataaggccagttatttaaagtta

SEQ ID NO: 16 - DNA, 32 bp, synthetic construct
  source 1..32: mol_type = other DNA
taactttaaataacgcctcttatttaaagtta

SEQ ID NO: 17 - DNA, 32 bp, synthetic construct
  source 1..32: mol_type = other DNA
taactttaaataagccccgttatttaaagtta

SEQ ID NO: 18 - DNA, 32 bp, synthetic construct
  source 1..32: mol_type = other DNA
taactttaaataagaccagttatttaaagtta

SEQ ID NO: 19 - DNA, 32 bp, synthetic construct
  source 1..32: mol_type = other DNA
taactttaaataaggcccgttatttaaagtta

SEQ ID NO: 20 - DNA, 32 bp, synthetic construct
  source 1..32: mol_type = other DNA
taactttaaataaggccggttatttaaagtta

SEQ ID NO: 21 - DNA, 34 bp, synthetic construct
  source 1..34: mol_type = other DNA
tcaatttctgagaactgtcattctcggaaattga

SEQ ID NO: 22 - DNA, 34 bp, synthetic construct
  source 1..34: mol_type = other DNA
ctcgtgtccgataactgtaattatcggacatgat

SEQ ID NO: 23 - DNA, 34 bp, synthetic construct
  source 1..34: mol_type = other DNA
aataggtctgagaacgcccattctcagacgtatt

SEQ ID NO: 24 - DNA, 34 bp, synthetic construct
  source 1..34: mol_type = other DNA
gaagttcctatacttctagaagaataggaacttc

(19) United States
(12) Patent Application Publication
(10) Pub. No.: US 2026/0103725 A1
(43) Pub. Date: Apr. 16, 2026

(54) GENETIC SWITCH

(71) Applicant: CASE WESTERN RESERVE UNIVERSITY, Cleveland, OH (US)

(72) Inventors: Ronald CONLON, Shaker Heights, OH (US); Mitchell DRUMM, Brecksville, OH (US); Kristian BAKER, Novelty, OH (US)

(21) Appl. No.: 19/358,004

(22) Filed: Oct. 14, 2025

Related U.S. Application Data
(60) Provisional application No. 63/707,067, filed on Oct. 14, 2024.
(60) Provisional application No. 63/852,294, filed on Jul. 28, 2025.

(51) Int. Cl. C12N 15/85

(52) U.S. Cl. CPC C12N 15/85; C12N 2800/30; C12N 2820/002

(57) ABSTRACT

A vector for gene expression comprises a promoter polynucleotide sequence, a polynucleotide sequence encoding a first detectable protein in a first reading frame, a polynucleotide sequence adapted for insertion of a test sequence, a nonsense-mediated decay polynucleotide sequence, a start codon that is in a second reading frame, a polynucleotide sequence encoding a second detectable protein in the first reading frame. A polynucleotide sequence encoding a third detectable protein is optionally present. The second reading frame may be one or two reading frames removed from the first reading frame. The polynucleotide sequence encoding a third detectable protein may be flanked on the 5 and 3′ ends by recombination sequences. A method of detectably inducing expression of a gene utilizes the vector.

### CROSS REFERENCE TO RELATED APPLICATIONS

This application claims benefit of pending U.S. Provisional Patent Application No. 63/707,067 filed on Oct. 14, 2024 and of U.S. Provisional Patent Application No. 63/852,294 filed on Jul. 28, 2025, the contents of which are incorporated by reference herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION BY REFERENCE OF MATERIAL SUBMITTED ELECTRONICALLY

The material contained in the Sequence Listing provided herewith in xml format in the XML file entitled “200512-00511.xml” created on Oct. 14, 2025 and containing 36,864 bytes, is hereby incorporated by reference herein.

### BACKGROUND OF THE INVENTION

This invention relates to an improved expression vector and method of controlling expression of gene products. More particularly, this invention relates to an expression vector containing a non-translatable nucleic acid portion, nonsense-mediated decay polynucleotide sequence, and an out-of-frame start codon. Even more particularly, this invention relates to an expression vector and method of verifying gene editing in stem cells using a Cre/loxP system to verify successful targeting of a specific type of cells.

The use of expression vectors in genetics research is well known and has been described in the prior art. Such expression vectors often comprise a polynucleotide having a promoter region, and an encoded translational stop which can be removed or suppressed, controlling the expression of one or more genes of interest or reporters.

For example, a representative expression system of the prior art is shown in FIG. 1. The system comprises a promoter sequence followed by the nucleic acid sequence encoding a protein such as green fluorescent protein, a “target” polynucleotide sequence, which is then followed by a further protein, such as a sequence encoding red fluorescent protein. In some cases, the red and green fluorescent proteins and might be substituted by other polypeptides or proteins of interest. Expression of the nucleic acid sequences from the promoter may be monitored by color detection of the red and/or green fluorescent proteins. If transcription occurs, the green fluorescent protein will be detectable. When some type of stop sequence is encoded into the target sequence, such as a stop codon, red fluorescent protein is typically greatly reduced. Removal or suppression of the stop sequence results in a strong signal from the red fluorescent protein. However, in prior expression systems, even in an uninduced or “off” state, red fluorescent protein will still be produced, although at a reduced level compared to the fully induced state in which the stop sequence is removed or suppressed. Such a state can be referred to as a “leaky” off state, because some expression “leaks” despite the stop sequence. This can result in switching that is between two different levels of an on state, rather than switching between a true off and an on state.

A switchable expression system which is not leaky in the “off” state has benefits. When the expressed sequences encode detectable proteins, low levels of expression in cells and tissues that should not express the gene product may hinder detection of a bona fide signal. It could also provide advantages where a gene product is toxic by limiting expression to a minimum, Therefore, there is a need for an improved genetic switch, in which the uninduced or “off” state is significantly more reduced and less “leaky” expression occurs in the “off” state.

It is also known that directed recombination can be achieved using a “Cre/loxP” system. In such a system, loxP sequences, typically short (13 base pairs) sequences which are palindromes of each other flank a region of nucleic acid to be modified. The Cre recombinase protein recognizes these loxP sequences and catalyzes a recombination at these locations. Using a Cre/loxP system can permit directed recombination in a wide variety of targeted cells such as stem cells of different tissues.

### BRIEF SUMMARY OF INVENTION

It is, therefore, an aspect of the present invention to provide an improved genetic switch.

It is another aspect of the present invention to provide a vector containing an improved genetic switch for polypeptide induction.

It is still another aspect of the present invention to provide a method of inducing gene expression from a vector containing the gene of interest.

It is yet another aspect of the present invention to provide a method of directing recombination to specific types of cells and verifying successful targeting of the intended cell types.

In general, the improved genetic switch of the present invention comprises a vector containing a promoter polynucleotide sequence, a polynucleotide sequence encoding a first detectable protein in a first reading frame, a polynucleotide sequence adapted for insertion of a test sequence which may encode a stop sequence, a nonsense-mediated decay polynucleotide sequence, a start codon that is not if the first reading frame, a polynucleotide sequence encoding a second detectable protein in the first reading frame. Additional polynucleotide sequences encoding other detectable proteins may be present. The detectable proteins may be replaced by other proteins or polypeptides of interest. A method of suppressing or removing the stop utilizes the vector.

In use, a gene of interest may be inserted into the vector to provide a polynucleotide comprising a promoter sequence, a sequence encoding a first protein, a sequence of interest or test sequence, a translation-inhibiting, nonsense-mediated decay region, a start codon that is not in the same reading frame as the prior sequence of interest, and a second protein.

The present invention further provides a vector for gene expression comprising a promoter polynucleotide sequence, a polynucleotide sequence encoding a first detectable protein, first and second complementary recombinase-target polynucleotide sequences (recombination sequences) flanking the polynucleotide sequence encoding a first detectable protein, a polynucleotide sequence encoding a second detectable protein, third and fourth complementary splice polynucleotide sequences flanking the polynucleotide sequence encoding a second detectable protein, and a polynucleotide sequence encoding a third detectable protein. The recombinase-target polynucleotide sequences may be selected from lox, rox and frt sequences for example or others known in the art.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 is a representation of a genetic switch system according to the prior art.

FIG. 2 is a representative comparison of the genetic switch of the prior art (top panel) and the present modification with the SQLCH component of the present invention (lower panels).

FIG. 3 is a representation of one example of a genetic switch using the SQLCH component in both an “off” state (left top) and “on” state (left bottom) and images of the bioluminescent signal provided for each of the detectable proteins encoded for in the construct (right side).

FIG. 4 is a representation of examples of a genetic switch using the SQLCH component in its entirety (top left), with the spacer removed (middle left) and with the intron deleted (bottom left) and corresponding images of the bioluminescent signal provided by akaluciferase (right side) for each example.

FIG. 5 is a representation of examples of a genetic switch illustrating the operation of the honeypot region of the SQLCH component.

FIG. 6 is a representation of examples of a genetic switch using the SQLCH component in with the honeypot region removed (top left), with the SQLCH component in its entirety (middle left), and with the SQLCH component and a further conversion of the Valine codon GTG to GTC and corresponding images of the bioluminescent signal provided by akaluciferase (right side) for each example.

FIG. 7 is a representation of examples of a genetic switch using the SQLCH component in its entirety (top left), and with components removed as noted and corresponding images of the bioluminescent signal provided for each of the detectable proteins encoded for in the construct (right side) for each example.

FIG. 8 is a representation of examples of a genetic switch using the SQLCH component using test sequences as noted and corresponding images of the bioluminescent signal provided for each of the detectable proteins encoded for in the constructs (right side) for each example.

FIG. 9 is a representation of examples of a genetic switch using the SQLCH component using test sequences with each of the detectable proteins replaced by non-detectable protein sequences as noted and corresponding images of the bioluminescent signal provided for each of the detectable proteins tested for in the constructs (right side) for each example.

FIG. 10 is a representation of examples of a genetic switch using the SQLCH component using test sequences as noted and corresponding images of the in vivo bioluminescent signal provided for each of the detectable proteins encoded for in the constructs (right side) for each example.

FIG. 11 is a graphic representation of the bioluminescent signal strength showing the difference in signal provided by SQLCH in the on state (left panel) and off state (right panel).

FIG. 12 is a representation of the use of a Cre/loxP vector in gene therapy.

FIG. 13 is a representation of a therapy vector combining Cre/loxP system in the SQLCH vector of the present invention.

FIG. 14 is a representation of the varying signals that can be produced using the SQLCH vector containing a Cre/loxP switch.

FIG. 15 is a schematic drawing of a reporter for gene therapy vector insertion and the resulting fluorescent signals.

FIG. 16 is a representation of a stem cell-specific gene therapy reporter of the present invention.

FIG. 17 is a representation of a stem cell specific Dre-delivery reporter (Panel A) and the resulting expression of the markers contained in the reporter with activation of Dre and/or Cre in Dre-delivered and non-Dre delivered stem cells and non-stem cells.

FIG. 18 is a representation of a method of detecting corrected stem cells and their descendants using the reporter of FIG. 17.

FIG. 19 is a representation of an example of the gene therapy reporter of the present invention utilizing a wild type CTFR gene in a donor vector to overcome a R553X mutation in a host CTFR gene in mouse lung, including an image of a subject mouse and a closer photograph of the lung tissue.

FIG. 20 is a series of photographs showing the signal provided by the gene therapy vector of the present invention in suppressing the nonsense mutation R553X by SMG1i in transiently transfected HEK293 cells.

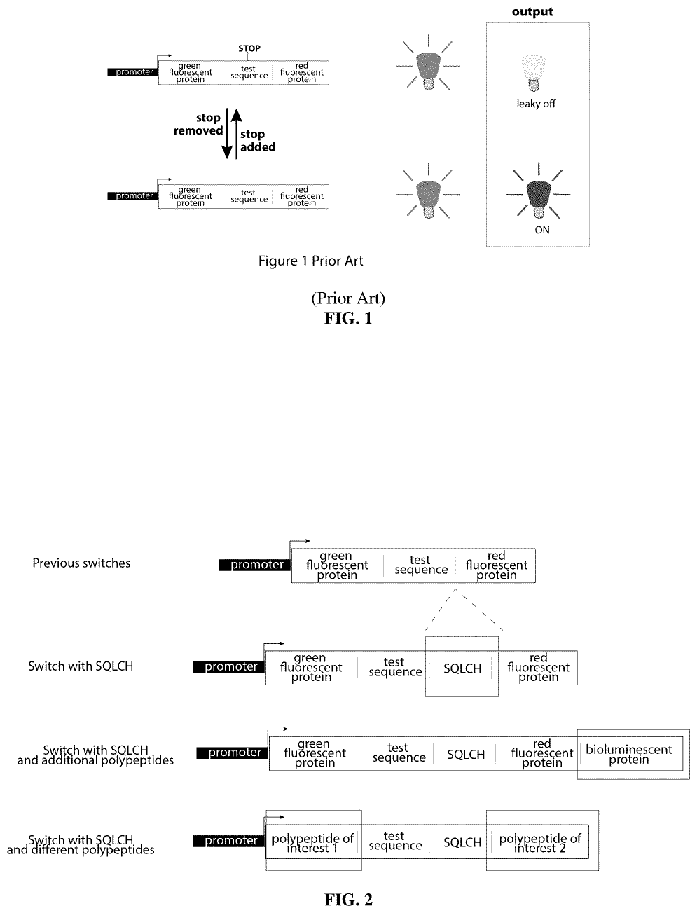

### DETAILED DESCRIPTION OF THE INVENTION

The present invention is directed toward an improved genetic switch. The following examples should not be viewed as limiting the scope of the invention. The claims will serve to define the inventions.

The improved genetic switch of the present invention includes a polynucleotide which can be inserted into previous expression systems. The previous expression system, for example, may comprise a promoter sequence, a sequence encoding a first detectable protein, one or more restriction endonuclease sites or similar sequences for insertion of a polynucleotide sequence of interest (or test sequence) and a second detectable protein. Optionally, a further detectable protein, such as a bioluminescent protein may be added to the 3′ end of the polynucleotide after the second detectable protein sequence or after the further detectable protein. The improvement on the genetic switch of the invention, named “SQLCH,” is shown in FIG. 2 and comprises a translation-inhibiting region, a start codon that is not in the same reading frame as the prior sequence of interest. The SQLCH sequence can be inserted immediately 3′ of the test sequence and comprises two regions. The first region is a stop codon, spacer or intron. The second region is an out-of-frame start codon. The first region decreases leaks by decreasing messenger RNA (mRNA) levels by nonsense-mediated decay (NMD). The second region or “honeypot” further lowers mRNA levels by diverting transcriptional reinitiation away from the downstream in-frame translational product, i.e., the sequence of the second detectable protein.

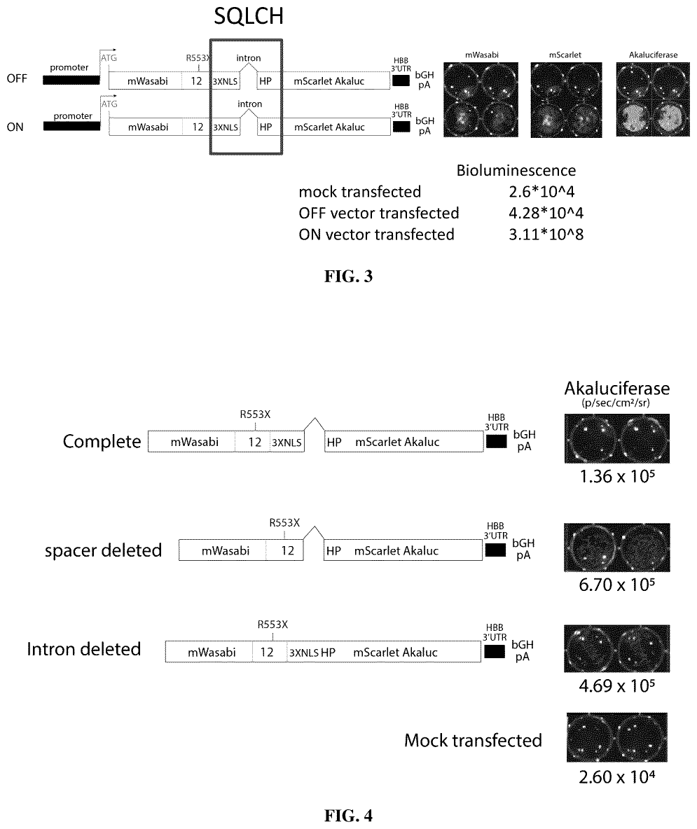

One particular embodiment of the improved genetic switch of the present invention is shown in FIG. 3. The polynucleotide sequence comprises a promoter region, a sequence encoding a first detectable protein (mWasabi) in a first open reading frame (frame 1), a test sequence which comprises 90 base pairs of the human cystic fibrosis transmembrane conductance regulator gene (12), the 3XNLS spacer sequence, an intron sequence, a “honeypot” region (HP) which contains a start sequence in reading frame 3 rather than frame 1, and a sequence encoding a second detectable protein and a further bioluminescent protein (mScarlet Akaluc). The test sequence contains a stop codon which causes termination prematurely. This mutation is implicated in cystic fibrosis and can be overridden by gene editing. Constructs containing the cystic fibrosis-causing mutation were tested in transiently transfected human cells in culture in an “off” or unsuppressed state (top) and in an “on” or mutation-suppressing state (bottom). A difference in bioluminescent signal strength of 10,000 fold was observed.

The contribution of the NMD region to decreasing “leaky” expression in an off state is illustrated in FIG. 4. In that embodiment, the complete construct, the construct with the 3XNLS spacer removed, and the construct with the intron removed are provided. Mock transfected cells are shown as a control. FIG. 4 shows that the NMD region lowers leaks by 5-fold.

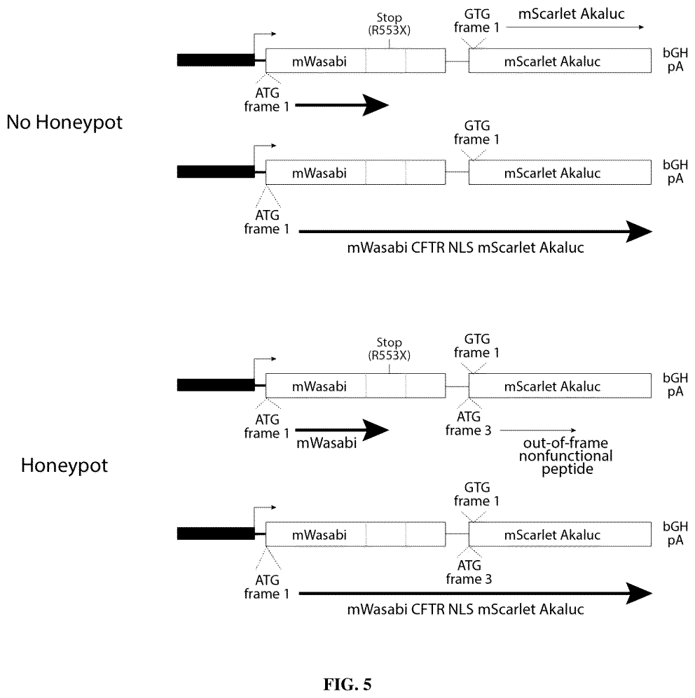

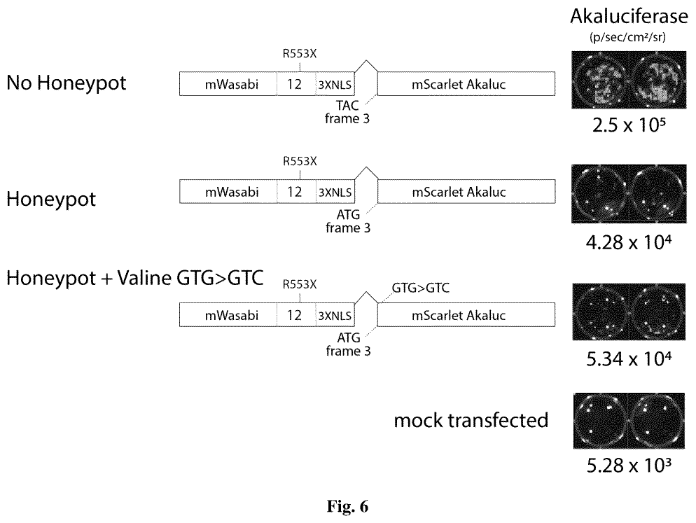

The operation of the honeypot region is shown in FIGS. 5 and 6. The stop codon in the test sequence will cause transcription to stop, but at a low level, transcription may otherwise reinitiate for the second detectable protein sequence. With the frameshift introduced by the honeypot, in-frame reinitiation and transcription of a full length mRNA is greatly reduced if not prevented for the second detectable protein (mScarlet Akaluc). When the stop codon in the test sequence is suppressed, transcription proceeds in-frame through the honeypot region, resulting in a full-length mRNA and resulting polypeptide. The out-of-frame start codon does not come into play where transcription continues through the end of the test sequence and into the second detectable protein sequence. In the examples shown, the frame shift in the “honeypot” region is from frame 1 to frame 3, although a frame shift from frame 1 to frame 2 has also been found to function (data not shown). This is important because frame 2 might be thought to be distinct from frame 2 due to the interaction of codons between the reading frames. Where an ATG codon (start codon) is present in frame 1 encoding methionine, and the immediately following nucleotide is an A, the result in frame 2 is a TGA or stop codon. This might be thought of as resulting in frame 2 not providing a long open reading frame. However, a shift from frame 1 to frame 2 works equally as well as a shift from frame 1 to frame 3.

In the constructs shown in FIG. 6, a 6-fold decrease in leaky expression of luciferase signal is shown, with a mock transfected sample control. Without the honeypot (top), the codon in frame 3 at the beginning of the mScarlet Akaluc gene is TAC. With the honeypot (middle), the codon in frame 3 at the beginning of mScarlet Akaluc is the start codon ATG. A significantly reduced signal is observed over the construct with no honey pot. Similar results are observed where the honeypot is present and a further modification of the Valine GTG codon to GTC is provided.

Human culture cells were transiently transfected to demonstrate the effects of removal of portions of the NMD. Constructs and results are provided in FIG. 7.

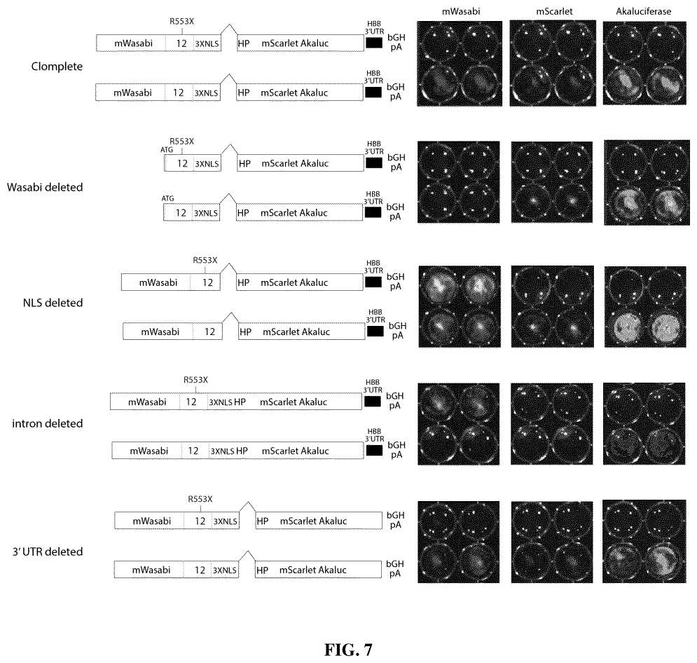

FIG. 8 shows that SQLCH will work with various test sequences. In FIG. 8, different cystic fibrosis-causing mutations were tested in transiently transfected human cells in culture in an “off” or unsuppressed state (top of each pair) and in an “on” or mutation-suppressing state (bottom of each pair).

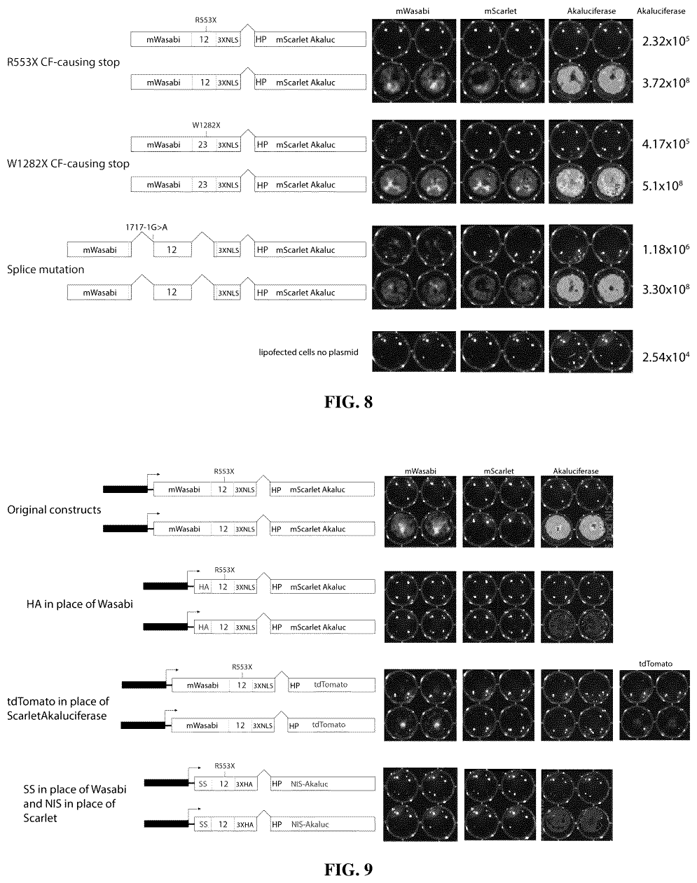

SQLCH will also work with different detectable proteins, as shown in FIG. 9. In FIG. 9, constructs containing variations of the detectable proteins were tested in transiently transfected human cells in culture in an “off” or unsuppressed state (top of each pair) and in an “on” or mutation-suppressing state (bottom of each pair).

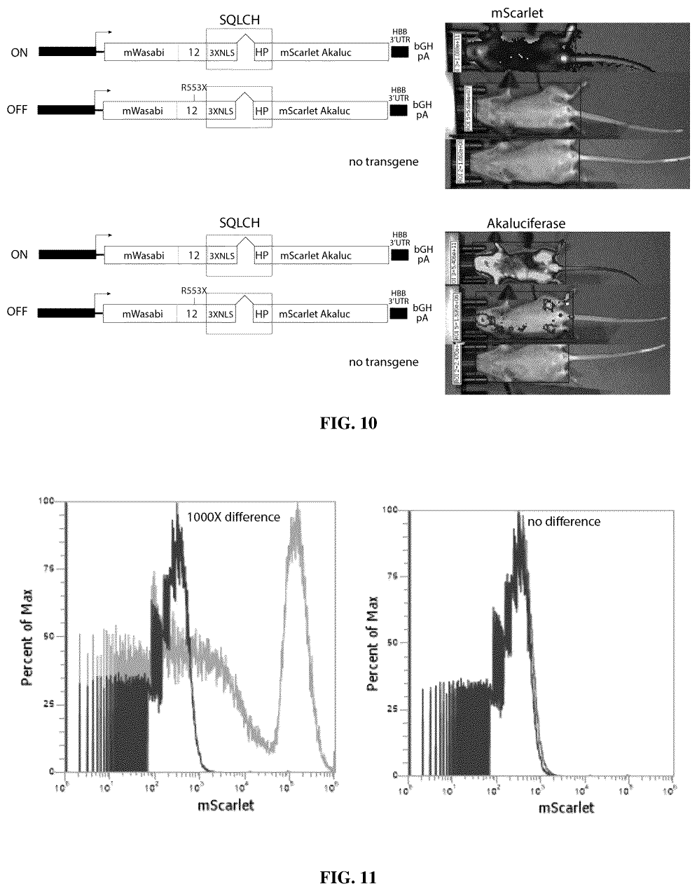

As stated above, the genetic switch of the present invention can be detected in vivo. The bioluminescent signals mScarlet (top) and Akaluciferase (bottom) of transgenic mice with SQLCH switches in the on and off conditions are shown in FIG. 10 with a non-transgenic control (bottom). A 10,000 fold difference in signal strength between on and off states was observed for mScarlet and a 100,000 fold difference was observed for Akaluciferase. The signal can also be specific to a tissue or organ.

FIG. 11 provides a graphic representation quantifying the difference in signal provided by SQLCH in the on and off state. No difference is observed in mouse fibroblast cells between the negative control and the off state (right panel) but a 1000-fold difference in signal is observed between the on and off states (left panel).

In a further example, the present invention may also incorporate a Cre/loxP switch to detect incorporation of a desired sequence into a specific type of cell, such as stem cells, particularly to detect bioluminescence in vivo. If for example, a reporter gene can only be expressed in stem cells, any bioluminescence detected in vivo can reasonably be assumed to result from successful targeting of stem cells with the construct. This provides the further advantages of greater confidence in determining whether a gene editing or therapy attempt was successful in the target cells, and the ability to screen multiple candidate therapies at one time in vivo.

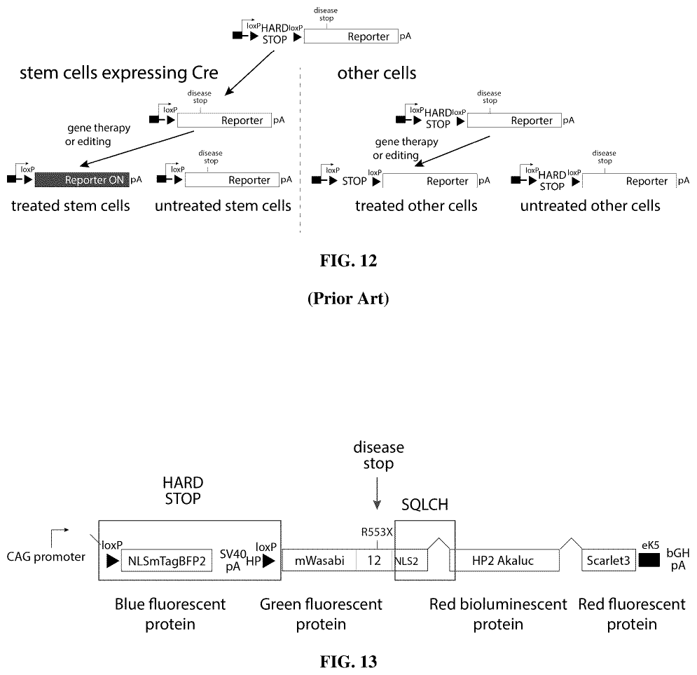

In such an example, the loxP sequences can be inserted into the genome flanking a hard stop sequence, and the cell supplied with or induced to express the Cre recombinase protein. When Cre excises the region containing the hard stop sequence between the lox P sequences a permanent change is made in the genome of that cell and all its daughter cells. The principle of use of a Cre/loxP system to detect bioluminescence in successfully targeted stem cell is provided in FIG. 12. Only incorporation of the reporter gene into stem cells will result in bioluminescence from expression of the reporter.

In the variation of the present invention additionally using a Cre/loxP switch, an additional “hard stop” element is inserted 5′ to the previously described construct, as shown in FIG. 13. The added sequence comprises loxP sequences flanking a nuclear localization sequences (NLS) and an additional different marker, such as mTAGBFP2 (blue fluorescent protein). Additional sequences may be added as needed such as a promoter sequence on the 5′end and a polyA sequence following the marker sequence.

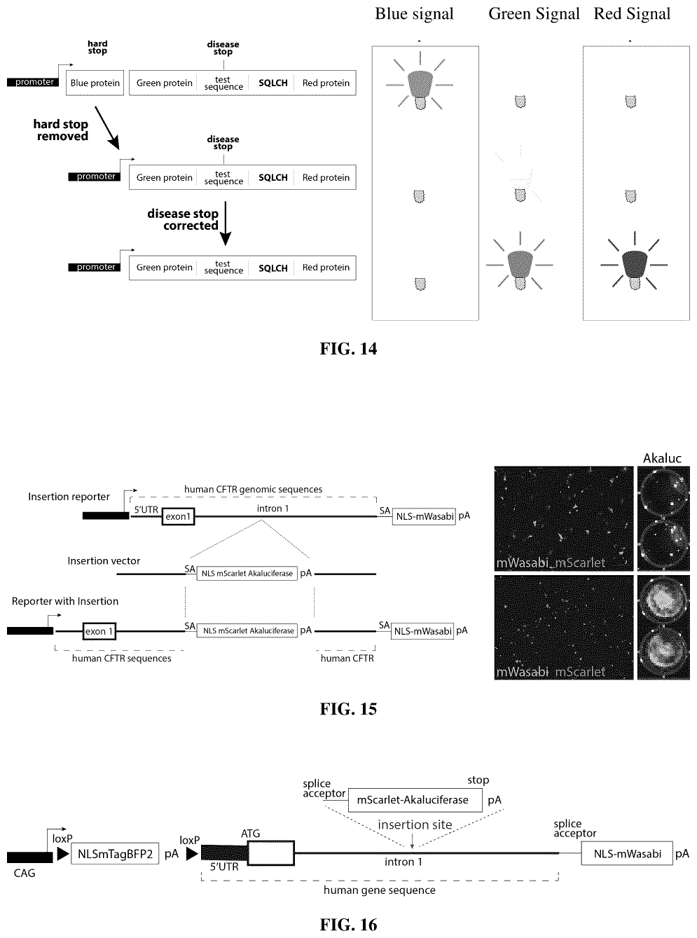

The variable signals that can be provided are illustrated in FIG. 14. When the complete construct is present, only the first fluorescent protein is observed—in this case a blue fluorescent signal. When the hard stop region is deleted, such as by administration or expression of Cre and recombination at the loxP sequences, a weak signal from the second protein (green signal) may be observed. When a stop in the test sequence is additionally corrected, both the second (in this example, green) and a third (red) signal is observed. It is envisioned that various genetic diseases affected by stem cells could be treated with the present invention, are listed in Table 1 and the genes implicated in those diseases are listed in Table 2.

This may be put into application as shown generally in FIG. 15. In this example, An insertion reporter contains the human CFTR exon 1 and intron 1 followed by a Splice Acceptor (SA) sequence, which is then followed by a fluorescent protein gene (NLS-mWasabi) and a poly adenylation (pA) sequence. This construct expresses the green mWasabi protein. An insertion vector having a second SA sequence 5′ to a second marker protein sequence (NLS mScarlet Akaluciferase) is inserted into intron 1. The resulting construct expresses both MScarlet and Akaluciferase.

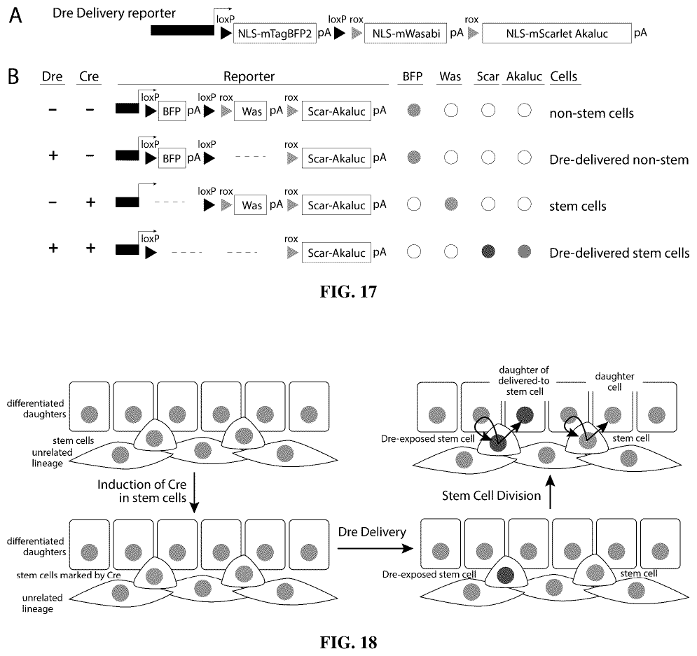

A further example, representing a stem cell-specific gene therapy reporter, is provided in FIG. 16. The present invention may also include a Dre-rox recombination system, and a recombination system similar to the Cre/loxP system. A stem cell specific Dre-delivery reporter is provided in FIG. 17, Panel A and expression of the markers contained in the reporter with activation of Dre and/or Cre in Dre-delivered and non-Dre delivered stem cells and non-stem cells are provided in FIG. 17, Panel B. Dre-delivered and non-Dre-delivered non-stem cells only expressed Blue Fluorescent Protein (BFP). Non-Dre-delivered stem cells only expressed the mWasabi protein, and Dre-delivered stem cells expressed both mScarlet and Akaluciferase. FIG. 18 is a representation of a method of detecting corrected stem cells and their descendants using the reporter of FIG. 17. Treated stem cells are induced to express Cre, activating Cre/loxP recombination and inducing expression of BFP. Dre delivery to the stem cells then results in activation of the Dre-rox system and expression of mScarlet in Dre-exposed stem cells. Expression of either BFP or mScarlet is exhibited in progeny cells of the treated stem cells. It is envisioned that various genetic diseases affected by stem cells could be treated with the present invention, are listed in Table 1 and the genes implicated in those diseases are listed in Table 2.

TABLE 1

| Stem Cell | Inducible Cre allele | Genetic Diseases |
|---|---|---|
| muscle (satellite) | Pax7^{tm1(Cre/ERT2)Gaka} | DMD/BMD, CMD, |
| | | LGMD |
| upper airway epithelium | Krt5^{tm1.1(Cre/ERT2)Blh} | CF, PCD |
| lower airway epithelium | Sftp^{tm1(Cre/ERT2)Blh} | SDD, PAM |
| epidermis | Lgr6^{tm2.1(Cre/ERT2)Cle} | DEB, EBS, JEB |
| intestinal epithelium | Lgr5^{tm1(Cre/ERT2)Cle} | CoDEs, CF, MVID |

TABLE 2

| Disease | Gene(s) |
|---|---|
| DMD/BMD | DMD |
| CMD | LAMA2, COL6A1, COL6A2, COL6A3 |
| LGMD | CAPN3, SGCA, SGCB, SGCG, SGCD, DYSF, TTN |
| CF | CFTR |
| PCD | DNAH5, DNAH9, DNAH11, CCDC103, ODAD1, |
| | ODAD2, ODAD3, ODAD4, DNAI1, DNAI2, NME8, |
| | DNAL1, CFAP298, CFAP300, DNAAF1, DNAAF2, |
| | DNAAF3, DNAAF4, DNAAF5, DNAAF6, DNAAF11 |
| SDD | SFTPB, SFTPC, ABCA3, NKX2.1 |
| PAM | SLC34A2 |
| DEB | COL7A1 |
| EBS | KRT5, KRT14, PLEC |
| JEB | LAMA3, LAMB3, LAMC2, COL17A1 |
| CoDE | AGR2, APOB, DGAT1, GUCY2C, LCT, SAR1B, SI, |
| | SLC10A2, SLC26A3, SLC39A4, SLC51B, SLC5A1, |
| | SLC7A7, SLC9A3, TREH |
| MVID | MYO5B |

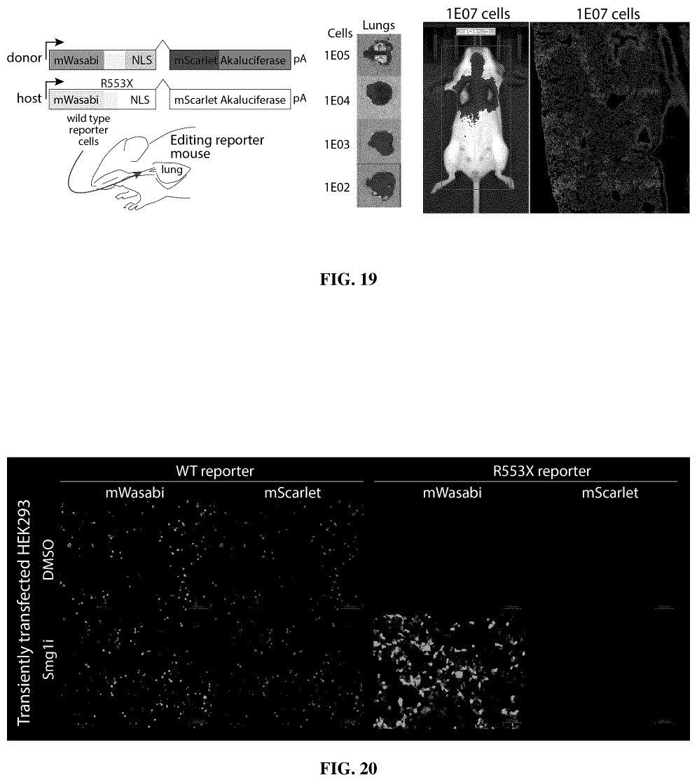

The sensitivity of the present invention for providing a detectable signal of successful delivery of a desired gene to target cells is shown in FIG. 19. A gene reporter vector containing the R553X mutation of the CFTR gene was present in host mice. The R553X mutation is a stop mutation which causes premature transcription of the CFTR gene, causing a truncated protein CTFR protein or no protein to be formed. As illustrated in FIG. 19, the expression of the mWasabi gene occurs at a low level and no expression of the mScarlet Akaluciferase gene occurs due to the presence of the NLS spacer sequence and the following “honeypot” sequence (\hat{ }). Donor cells with the reporter without the R553X mutation were provided to the lungs of host mice by transplantation with a bronchoscope into lung bronchi. Because of the transcription of the full gene transplanted into the donor mice, transcription can proceed through the mScarlet Akaluciferase gene and the mScarlet Akaluciferase protein is expressed and provides an observable signal in cells in which the gene therapy vector has been inserted. Based on an estimate of 10⁸ lung cells being present in a 30 g mouse, a signal representing as low as 10 percent (10⁷ cells) successful insertion of the gene therapy vector is observed.

The gene therapy vector of the present invention may also be used to screen nonsense mutation suppressing drugs. For example, SMG1i is recognized to suppress the nonsense mediated decay that is associated with some cases of cystic fibrosis. As shown in FIG. 20, HEK 293 cells were transiently transfected with a gene therapy vector containing either a wild type CTFR gene or a R553X-containing CTFR gene. The transiently transfected cells were treated either with SMG1i or DMSO as a control. In the wild type transfected cells, treatment with SMG1i provides no increase in expression of mWasabi because there is no nonsense mutation to suppress. However, in the R553X-containing cells, administration of SMG1i provides a strong signal from mWasabi while DMSO induces essentially no signal. It is envisioned that the gene therapy vector of the present invention may be used with other nonsense mutations and corresponding inhibitors or inhibitor candidates.

As presented herein, by “substantially identical” is meant that polynucleotide regions have sufficient homology with the named segments of DNA as to be able to hybridize under stringent conditions

In the sequences described herein, nucleotides should be understood to be represented by their standard one letter abbreviations: A for adenine, G for guanine, C for cytosine, T for thymine and U for uracil. The presence of a mixture of nucleotides may also be indicated with an abbreviation as recognized in the art: N for any base (A,C,G or T/U), R for purine (G or A), Y for pyrimidine (T/U or C), M for amino (A or C), K for keto (G or T/U), S for G or C, W for A or T/U, V for nucleotides other than T (A, C, or G), D for nucleotides other than C (A, G, or T), B for nucleotides other than A (C, G, or T), and H for nucleotides other than G (A, C, or T).

The word “comprising” and forms of the word “comprising” as used in this description and in the claims does not limit the invention claimed to exclude any variants or additions.

Research for this invention was supported by awards from the Cystic Fibrosis Foundation.

Based upon the foregoing disclosure, it should now be apparent that the present invention will carry out the objects set forth hereinabove. It is, therefore, to be understood that any variations evident fall within the scope of the claimed invention and thus, the selection of specific component elements can be determined without departing from the spirit of the invention herein disclosed and described.

SEQUENCES

R553X editing reporter (SEQ ID NO: 1)
tgttttggttggcgtaaggcgcctgtcagttaacggcagccggagtgcgcagccgccggcagcctcgctctgcccactgggtggggcgggagg
taggtggggtgaggcgagctggacgtgcgggcgcggtcggcctctggcggggcgggggaggggagggagggtcagcgaaagtagctcgc
gcgcgagcggccgcccaccctccccttcctctgggggagtcgttttacccgccgccggccgggcctcgtcgtctgattggctctcggggcccag
aaaactggcccttgccattggctcgtgttcgtgcaagttgagtccatccgccggccagcgggggcggcgaggaggcgctcccaggttccggcc
ctcccctcggccccgcgccgcagagtctggccgcgcgcccctgcgcaacgtggcaggaagcgcgcgctgggggggggacgggcagtag
ggctgagcggctgcggggcgggtgcaagcacgtttccgacttgagttgcctcaagaggggcgtgctgagccagacctccatcgcgcactccg
gggagtggagggaaggagcgagggctcagttgggctgttttggaggcaggaagcacttgctctcccaaagtcgctctgagttgttatcagtaag
ggagctgcagtggagtaggcggggagaaggccgcacccttctccggaggggggaggggagtgttgcaatacctttctgggagttctctgctgc
ctcctggcttctgaggaccgccctgggcctgggagaatcccttccccctcttccctcgtgatctgcaactccagtctttcttctgtagggcgcagtag
tccagggtctcgacattgattattgactagttattaatagtaatcaattacggggtcattagttcatagcccatatatggagttccgcgttacataactta
cggtaaatggcccgcctggctgaccgcccaacgacccccgcccattgacgtcaataatgacgtatgttcccatagtaacgccaatagggactttc
cattgacgtcaatgggtggagtatttacggtaaactgcccacttggcagtacatcaagtgtatcatatgccaagtacgccccctattgacgtcaatga
cggtaaatggcccgcctggcattatgcccagtacatgaccttatgggactttcctacttggcagtacatctacgtattagtcatcgctattaccatggt
cgaggtgagccccacgttctgcttcactctccccatctcccccccctccccacccccaattttgtatttatttattttttaattattttgtgcagcgatggg
ggcggggggggggggggggcgcgcgccaggcggggcggggcggggcgaggggggggcggggcgaggcggagaggtgcggcgg
cagccaatcagagcggcgcgctccgaaagtttccttttatggcgaggcggcggcggcggcggccctataaaaagcgaagcgcgcggcgggc
gggagtcgctgcgcgctgccttcgccccgtgccccgctccgccgccgcctcgcgccgcccgccccggctctgactgaccgcgttactcccaca
ggtgagcggggggacggcccttctcctccgggctgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgtggggagcgccgcgtgcggctccgcgct
gcccggcggctgtgagcgctgcgggcgcggcgcggggctttgtgcgctccgcagtgtgcgcgaggggagcgcggccgggggcggtgccc
cgcggtgcggggggggctgcgaggggaacaaaggctgcgtgcggggtgtgtgcgtgggggggtgagcagggggtgtgggcgcgtcggtc
gggctgcaaccccccctgcacccccctccccgagttgctgagcacggcccggcttcgggtgcggggctccgtacggggcgtggcgcggggc
tcgccgtgccgggcggggggggcggcagggggggtgccgggcggggggggccgcctcgggccggggagggctcgggggagggg
cgcggcggcccccggagcgccggcggctgtcgaggcgcggcgagccgcagccattgccttttatggtaatcgtgcgagagggcgcaggga
cttcctttgtcccaaatctgtgcggagccgaaatctgggaggcgccgccgcaccccctctagcgggcgcggggcgaagcggtgcggcgccgg
caggaaggaaatgggcggggagggccttcgtgcgtcgccgcgccgccgtccccttctccctctccagcctcggggctgtccgcggggggac
ggctgccttcgggggggacggggcagggggggttcggcttctggcgtgtgaccggcggctctagagcctctgctaaccatgttcatgccttctt
ctttttcctacagctcctgggcaacgtgctggttattgtgctgtctcatcattttggcaaagaattgcaagtttgtacaaaaaagcaggctgccaccatg
gtgagcaagggcgaggagaccaccatgggcgtgatcaagcccgacatgaagatcaagctgaagatggagggcaacgtgaacggccacgcc
ttcgtgatcgagggcgagggcgagggcaagccctacgatggcaccaacaccatcaacctggaggtgaaggagggcgcccccctgcccttca
gctacgacatcctgaccaccgccttcagctacggcaacagggccttcaccaagtaccctgacgacatccccaactacttcaagcagagcttcccc
gagggctacagctgggagaggaccatgaccttcgaggacaagggcatcgtgaaggtgaagagcgacatcagcatggaggaggacagcttca
tctacgagatccacctgaagggcgagaacttcccccccaacggccccgtgatgcagaaggagaccaccggctgggacgccagcaccgagag
gatgtacgtgagggacggggtgctgaagggggacgtgaagatgaagctgctgctggagggcggcggccaccacagggtggacttcaagacc
atctacagggccaagaaggccgtgaagctgcccgactaccacttcgtggaccacaggatcgagatcctgaaccacgacaaggactacaacaa
ggtgaccgtgtacgagatcgcagtggccaggaacagcaccgatggcatggacgagctgtacaaggacatctccaagtttgcagagaaagaca
atatagttcttggagaaggtggaatcacactgagtggaggtcaatgagcaagaatttctttagcaggtagcggaggaggaggtcccaagaagaa
gaggaaggtcgaccccaagaagaagaggaaggtcgaccccaagaagaagaggaaggtcagggtgagtctatgggacgcttgatgttttctttc
cccttcgagtccaagctaggcccttttgctaatcatgttcatacctcttatcttcctcccacagctccagccaccatggagcggaggaggagtgagc
aagggcgaggccgtgatcaaggagttcatgaggttcaaggtgcacatggagggcagcatgaacggccacgagttcgagatcgagggcgagg
gcgagggcaggccctacgagggcacccagaccgccaagctgaaggtgaccaagggggccccctgcccttcagctgggacatcctgagcc
cccagttcatgtacggcagcagggccttcaccaagcaccccgccgacatccccgactactacaagcagagcttccccgagggcttcaagtggg
agagggtgatgaacttcgaggacggcggcgccgtgaccgtgacccaggacaccagcctggaggacggcaccctgatctacaaggtgaagct
gaggggcaccaacttcccccccgacggccccgtgatgcagaagaagaccatgggctgggaggccagcaccgagaggctgtaccccgagga
cggcgtgctgaagggcgacatcaagatggccctgaggctgaaggacggcggcaggtacctggccgacttcaagaccacctacaaggccaag
aagcccgtgcagatgcccggcgcctacaacgtggacaggaagctggacatcaccagccacaacgaggactacaccgtggtggagcagtacg
agaggagcgagggcaggcacagcaccggcggcatggacgagctgtacaaggaggacgccaagaacatcaagaagggccccgcccccttc
taccccctggaggatggcaccgcaggcgagcagctgcacaaggccatgaagaggtacgccctggtgcccggggccatcgccttcaccgacg
cccacatccaggtggacgtgacctacgcagagtacttcgagatgagcgtgaggctggccgaggccatgaggaggtacggcctgaacaccaac
cacaggatcgtggtgtgcagcgagaacagcagccagttcttcatgcccgtgctgggcgccctgttcatcggggggccgtggcccccgccaac
gacatctacaacgagagggagctgctgaacagcatgggcatcagccagcccaccgtggtgttcgtgagcaagaagggcctgaggaaggtgct
gaacgtgcagaagaagctgcccatcatcaggaagatcatcatcatggacagcaagaccgactaccagggcttccagagcatgtacaccttcgtg
accagccacctgccccccagcttcaacgagtatgacttcgtgcccgagagcttcgacagggacaagaccatcgccctgatcatgaacagcagc
ggcagcaccggcctgcccaagggcgtggccctgccccacaggaccgcctgcgtgaggttcagccacgccagggaccccatcttcggctacc
agaacatccccgacaccgccatcctgagcgtggtgcccttccaccacggcttcggcatgttcaccaccctgggctacctgatctgcggcttcagg
gtggtgctgatgtacaggttcgaggaggagctgttcctgaggagcctgcaggactacaagatccagagcgccctgctggtgcccaccctgttca
gctgcctggccaagagcaccctgatcgacaagtacgacctgagcagcctgagggagatcgcttcaggtggcgcccccctgagcaaggaggtg
ggcgaggccgtggccaagaggttcaggctgcccggcatcaggcagggctacggcctgaccgagaccaccaacgccgtgatgatcacccccg
agggcgacaggaagcccggcagcgtgggcaaggtggtgcccttcttcgaggccaaggtggtggacctggtgaccggcaagaccctgggcgt
gaaccagaggggcgagctgtgcgtgaggggccccatgatcatgagcggctacgtgaacaaccccgaggccaccaacgccctgatcgacaag
gacggctggctgcacagcggcgacatcgcctactgggacgaggacgagcacttcttcatcgtggacaggctgaagagcctgatcaagtacaag
ggctaccaggtggcccccgccgagctggagggcatcctgctgcagcacccctacatctttgacgcaggcgtggccggcctgccagatgacga
cgcaggagagctgcccgcagctgtggtggtgctggagcacggcaagaccatgaccgagaaggagatcgtggactacgtggccagccaggtg
accaccgccaagaagctgaggggaggcgtggtgttcgtggacgaggtgcccaggggcagcaccggcaagctggacgccaggaagatcag
ggagatcctgaccaaggccaagaaggacggcaagatcgccgtgtaacgctttcttgctgtccaatttctattaaaggttcctttgttccctaagtcca
actactaaactgggggatattatgaagggccttgagcatctggattctgcctacccagctttcttgtacaaagtggctgtgccttctagttgccagcca
tctgttgtttgcccctcccccgtgccttccttgaccctggaaggtgccactcccactgtcctttcctaataaaatgaggaaattgcatcgcattgtctga
gtaggtgtcattctattctggggggtggggggggcaggacagcaagggggaggattgggaagacaatagcaggcatgctggggatgcggtg
ggctctatggtgggcgggagtcttctgggcaggcttaaaggctaacctggtgtgtgggcgttgtcctgcaggggaattgaacaggtgtaaaattg
gagggacaagacttcccacagattttcggttttgtcgggaagttttttaataggggcaaataaggaaaatgggaggataggtagtcatctggggtttt
atgcagcaaaactacaggttattattgcttgtgatccgcctcggagtattttccatcgaggtagattaaagacatgctcacccgagttttatactctcct
gcttgagatccttactacagtatgaaattacagtgtcgcgagttagactatgtaagcagaattttaatcatttttaaagagcccagtacttcatatccattt
ctcccgctccttctgcagccttatcaaaaggtattttagaacactcattttagccccattttcatttattatactggcttatccaacccctagacagagcat
tggcattttccctttcctgatcttagaagtctgatgactcatgaaaccagacagattagttacatacaccacaaatcgaggctgtagctggggcctca
acactgcagttcttttataactccttagtacactttttgttgatcctttgccttgatccttaattttcagtgtctatcacctctcccgtcaggtggtgttccaca
tttgggcctattctcagtccagggagttttacaacaatagatgtattgagaatccaacctaaagcttaactttccactcccatgaatgcctctctccttttt
ctccatttataaactgagct
Spacer-recoded 3X SV40 NLS (SEQ ID NO: 2)
ggtagcggaggaggaggtcccaagaagaagaggaaggtcgaccccaagaagaagaggaaggtcgaccccaagaagaagaggaaggtc
Spacer-recoded to remove potential stem-loop 3X SV40 NLS (SEQ ID NO: 3)
ggtagcggaggaggaggtcccaagaagaagaggaaagttgatccaaagaagaagaggaaagttgacccaaagaagaagaggaaggtc
Spacer-recoded 3X HA (SEQ ID NO: 4)
Tacccctacgacgtgcccgactacgcctacccctacgacgtgcccgactacgcctacccctacgacgtgcccgactacgcc
human beta globin minimal intron 2 (SEQ ID NO: 5)
agggtgagtctatgggacgcttgatgttttctttccccttcgagtccaagctaggcccttttgctaatcatgttcatacctcttatcttcctcccacagctc
human beta globin intron 1 (SEQ ID NO: 6)
ggcaggttggtatcaaggttacaagacaggtttaaggagaccaatagaaactgggcatgtggagacagagaagactcttgggtttctgataggca
ctgactctctctgcctattggtctattttcccacccttaggctg
honeypot 1 (SEQ ID NO: 7)
cagccaccatggagcggaggagga
honeypot 2 (SEQ ID NO: 8)
cagccaccatggtcaagatccacgagcggaggagga
frame 2 honeypot (SEQ ID NO: 9)
cagaaccaccatggagccggaggagga
wild type control editing reporter (SEQ ID NO: 10)
tgttttggttggcgtaaggcgcctgtcagttaacggcagccggagtgcgcagccgccggcagcctcgctctgcccactgggtggggcgggagg
taggtggggtgaggcgagctggacgtgcgggcgcggtcggcctctggcggggcgggggaggggagggagggtcagcgaaagtagctcgc
gcgcgagcggccgcccaccctccccttcctctgggggagtcgttttacccgccgccggccgggcctcgtcgtctgattggctctcggggcccag
aaaactggcccttgccattggctcgtgttcgtgcaagttgagtccatccgccggccagcgggggcggcgaggaggcgctcccaggttccggcc
ctcccctcggccccgcgccgcagagtctggccgcgcgcccctgcgcaacgtggcaggaagcgcgcgctgggggggggacgggcagtag
ggctgagcggctgcgggggggtgcaagcacgtttccgacttgagttgcctcaagaggggcgtgctgagccagacctccatcgcgcactccg
gggagtggagggaaggagcgagggctcagttgggctgttttggaggcaggaagcacttgctctcccaaagtcgctctgagttgttatcagtaag
ggagctgcagtggagtaggcggggagaaggccgcacccttctccggaggggggaggggagtgttgcaatacctttctgggagttctctgctgc
ctcctggcttctgaggaccgccctgggcctgggagaatcccttccccctcttccctcgtgatctgcaactccagtctttcttctgtagggcgcagtag
tccagggtctcgacattgattattgactagttattaatagtaatcaattacggggtcattagttcatagcccatatatggagttccgcgttacataactta
cggtaaatggcccgcctggctgaccgcccaacgacccccgcccattgacgtcaataatgacgtatgttcccatagtaacgccaatagggactttc
cattgacgtcaatgggtggagtatttacggtaaactgcccacttggcagtacatcaagtgtatcatatgccaagtacgccccctattgacgtcaatga
cggtaaatggcccgcctggcattatgcccagtacatgaccttatgggactttcctacttggcagtacatctacgtattagtcatcgctattaccatggt
cgaggtgagccccacgttctgcttcactctccccatctcccccccctccccacccccaattttgtatttatttattttttaattattttgtgcagcgatggg
ggcggggggggggggggggcgcgcgccaggcggggcggggcggggcgaggggcggggcggggcgaggcggagaggtgcggcgg
cagccaatcagagcggcgcgctccgaaagtttccttttatggcgaggcggcggcggcggcggccctataaaaagcgaagcgcgcggcgggc
gggagtcgctgcgcgctgccttcgccccgtgccccgctccgccgccgcctcgcgccgcccgccccggctctgactgaccgcgttactcccaca
ggtgagcgggcgggacggcccttctcctccgggctgtaattagcgcttggtttaatgacggcttgtttcttttctgtggctgcgtgaaagccttgagg
ggctccgggagggccctttgtgcggggggagcggctcggggggtgcgtgcgtgtgtgtgtgcgtggggagcgccgcgtgcggctccgcgct
gcccggcggctgtgagcgctgcgggcgcggcgcggggctttgtgcgctccgcagtgtgcgcgaggggagcgcggccgggggcggtgccc
cgcggtgcggggggggctgcgaggggaacaaaggctgcgtgcggggtgtgtgcgtggggggggagcagggggtgtgggcgcgtcggtc
gggctgcaaccccccctgcacccccctccccgagttgctgagcacggcccggcttcgggtgcggggctccgtacggggcgtggcgcggggc
tcgccgtgccgggggggggtggcggcaggtgggggtgccgggcggggcggggccgcctcgggccggggagggctcgggggagggg
cgcggcggcccccggagcgccggcggctgtcgaggcgcggcgagccgcagccattgccttttatggtaatcgtgcgagagggcgcaggga
cttcctttgtcccaaatctgtgcggagccgaaatctgggaggcgccgccgcaccccctctagcgggcgcggggcgaagcggtgcggcgccgg
caggaaggaaatgggcggggagggccttcgtgcgtcgccgcgccgccgtccccttctccctctccagcctcggggctgtccgcggggggac
ggctgccttcgggggggacggggcagggcggggttcggcttctggcgtgtgaccggcggctctagagcctctgctaaccatgttcatgccttctt
ctttttcctacagctcctgggcaacgtgctggttattgtgctgtctcatcattttggcaaagaattgcaagtttgtacaaaaaagcaggctgccaccatg
gtgagcaagggcgaggagaccaccatgggcgtgatcaagcccgacatgaagatcaagctgaagatggagggcaacgtgaacggccacgcc
ttcgtgatcgagggcgagggcgagggcaagccctacgatggcaccaacaccatcaacctggaggtgaaggagggcgcccccctgcccttca
gctacgacatcctgaccaccgccttcagctacggcaacagggccttcaccaagtaccctgacgacatccccaactacttcaagcagagcttcccc
gagggctacagctgggagaggaccatgaccttcgaggacaagggcatcgtgaaggtgaagagcgacatcagcatggaggaggacagcttca
tctacgagatccacctgaagggcgagaacttcccccccaacggccccgtgatgcagaaggagaccaccggctgggacgccagcaccgagag
gatgtacgtgagggacggggtgctgaagggggacgtgaagatgaagctgctgctggagggcggcggccaccacagggtggacttcaagacc
atctacagggccaagaaggccgtgaagctgcccgactaccacttcgtggaccacaggatcgagatcctgaaccacgacaaggactacaacaa
ggtgaccgtgtacgagatcgcagtggccaggaacagcaccgatggcatggacgagctgtacaaggacatctccaagtttgcagagaaagaca
atatagttcttggagaaggtggaatcacactgagtggaggtcaacgagcaagaatttctttagcaggtagcggaggaggaggtcccaagaagaa
gaggaaggtcgaccccaagaagaagaggaaggtcgaccccaagaagaagaggaaggtcagggtgagtctatgggacgcttgatgttttctttc
cccttcgagtccaagctaggcccttttgctaatcatgttcatacctcttatcttcctcccacagctccagccaccatggagcggaggaggagtgagc
aagggcgaggccgtgatcaaggagttcatgaggttcaaggtgcacatggagggcagcatgaacggccacgagttcgagatcgagggcgagg
gcgagggcaggccctacgagggcacccagaccgccaagctgaaggtgaccaagggcggccccctgcccttcagctgggacatcctgagcc
cccagttcatgtacggcagcagggccttcaccaagcaccccgccgacatccccgactactacaagcagagcttccccgagggcttcaagtggg
agagggtgatgaacttcgaggacggcggcgccgtgaccgtgacccaggacaccagcctggaggacggcaccctgatctacaaggtgaagct
gaggggcaccaacttcccccccgacggccccgtgatgcagaagaagaccatgggctgggaggccagcaccgagaggctgtaccccgagga
cggcgtgctgaagggcgacatcaagatggccctgaggctgaaggacggcggcaggtacctggccgacttcaagaccacctacaaggccaag
aagcccgtgcagatgcccggcgcctacaacgtggacaggaagctggacatcaccagccacaacgaggactacaccgtggtggagcagtacg
agaggagcgagggcaggcacagcaccggcggcatggacgagctgtacaaggaggacgccaagaacatcaagaagggccccgcccccttc
taccccctggaggatggcaccgcaggcgagcagctgcacaaggccatgaagaggtacgccctggtgcccggggccatcgccttcaccgacg
cccacatccaggtggacgtgacctacgcagagtacttcgagatgagcgtgaggctggccgaggccatgaggaggtacggcctgaacaccaac
cacaggatcgtggtgtgcagcgagaacagcagccagttcttcatgcccgtgctgggcgccctgttcatcggggggccgtggcccccgccaac
gacatctacaacgagagggagctgctgaacagcatgggcatcagccagcccaccgtggtgttcgtgagcaagaagggcctgaggaaggtgct
gaacgtgcagaagaagctgcccatcatcaggaagatcatcatcatggacagcaagaccgactaccagggcttccagagcatgtacaccttcgtg
accagccacctgccccccagcttcaacgagtatgacttcgtgcccgagagcttcgacagggacaagaccatcgccctgatcatgaacagcagc
ggcagcaccggcctgcccaagggcgtggccctgccccacaggaccgcctgcgtgaggttcagccacgccagggaccccatcttcggctacc
agaacatccccgacaccgccatcctgagcgtggtgcccttccaccacggcttcggcatgttcaccaccctgggctacctgatctgcggcttcagg
gtggtgctgatgtacaggttcgaggaggagctgttcctgaggagcctgcaggactacaagatccagagcgccctgctggtgcccaccctgttca
gctgcctggccaagagcaccctgatcgacaagtacgacctgagcagcctgagggagatcgcttcaggtggcgcccccctgagcaaggaggtg
ggcgaggccgtggccaagaggttcaggctgcccggcatcaggcagggctacggcctgaccgagaccaccaacgccgtgatgatcacccccg
agggcgacaggaagcccggcagcgtgggcaaggtggtgcccttcttcgaggccaaggtggtggacctggtgaccggcaagaccctgggcgt
gaaccagaggggcgagctgtgcgtgaggggccccatgatcatgagcggctacgtgaacaaccccgaggccaccaacgccctgatcgacaag
gacggctggctgcacagcggcgacatcgcctactgggacgaggacgagcacttcttcatcgtggacaggctgaagagcctgatcaagtacaag
ggctaccaggtggcccccgccgagctggagggcatcctgctgcagcacccctacatctttgacgcaggcgtggccggcctgccagatgacga
cgcaggagagctgcccgcagctgtggtggtgctggagcacggcaagaccatgaccgagaaggagatcgtggactacgtggccagccaggtg
accaccgccaagaagctgaggggaggcgtggtgttcgtggacgaggtgcccaggggcagcaccggcaagctggacgccaggaagatcag
ggagatcctgaccaaggccaagaaggacggcaagatcgccgtgtaacgctttcttgctgtccaatttctattaaaggttcctttgttccctaagtcca
actactaaactgggggatattatgaagggccttgagcatctggattctgcctacccagctttcttgtacaaagtggctgtgccttctagttgccagcca
tctgttgtttgcccctcccccgtgccttccttgaccctggaaggtgccactcccactgtcctttcctaataaaatgaggaaattgcatcgcattgtctga
gtaggtgtcattctattctggggggggggtggggcaggacagcaagggggaggattgggaagacaatagcaggcatgctggggatgcggtg
ggctctatggtgggcgggagtcttctgggcaggcttaaaggctaacctggtgtgtgggcgttgtcctgcaggggaattgaacaggtgtaaaattg
gagggacaagacttcccacagattttcggttttgtcgggaagttttttaataggggcaaataaggaaaatgggaggataggtagtcatctggggtttt
atgcagcaaaactacaggttattattgcttgtgatccgcctcggagtattttccatcgaggtagattaaagacatgctcacccgagttttatactctcct
gcttgagatccttactacagtatgaaattacagtgtcgcgagttagactatgtaagcagaattttaatcatttttaaagagcccagtacttcatatccattt
ctcccgctccttctgcagccttatcaaaaggtattttagaacactcattttagccccattttcatttattatactggcttatccaacccctagacagagcat
tggcattttccctttcctgatcttagaagtctgatgactcatgaaaccagacagattagttacatacaccacaaatcgaggctgtagctggggcctca
acactgcagttcttttataactccttagtacactttttgttgatcctttgccttgatccttaattttcagtgtctatcacctctcccgtcaggtggtgttccaca
tttgggcctattctcagtccagggagttttacaacaatagatgtattgagaatccaacctaaagcttaactttccactcccatgaatgcctctctccttttt
ctccatttataaactgagct

Example loxP Sites

| Name | 13 bp Recognition Region | 8 bp Spacer Region | 13 bp Recognition Region |
|---|---|---|---|
| Wild-Type | ATAACTTCGTATA | ATGTATGC | TATACGAAGTTAT |
| | (SEQ ID NO: 11) | | (SEQ ID NO: 13) |
| lox 511 | ATAACTTCGTATA | ATGTATaC | TATACGAAGTTAT |
| lox 5171 | ATAACTTCGTATA | ATGTgTaC | TATACGAAGTTAT |
| lox 2272 | ATAACTTCGTATA | AaGTATcC | TATACGAAGTTAT |
| M2 | ATAACTTCGTATA | AgaaAcca | TATACGAAGTTAT |
| M3 | ATAACTTCGTATA | taa TACCA | TATACGAAGTTAT |
| M7 | ATAACTTCGTATA | AgaTAGAA | TATACGAAGTTAT |
| M11 | ATAACTTCGTATA | cgaTAcca | TATACGAAGTTAT |
| lox 71 | TACCGTTCGTATA | NNNTANNN | TATACGAAGTTAT |
| | (SEQ ID NO: 12) | | |
| lox 66 | ATAACTTCGTATA | NNNTANNN | TATACGAACGGTA |
| loxPsym | ATAACTTCGTATA | atgtacat | TATACGAAGTTAT |

### Dre/rox

roxP
(SEQ ID NO: 14)
TAACTTTAAATAATGCCAATTATTTAAAGTTA
rox7
(SEQ ID NO: 15)
TAACTTTAAATAAGGCCAGTTATTTAAAGTTA
rox8
(SEQ ID NO: 16)
TAACTTTAAATAACGCCTCTTATTTAAAGTTA
rox9
(SEQ ID NO: 17)
TAACTTTAAATAAGCCCCGTTATTTAAAGTTA
rox34
(SEQ ID NO: 18)
TAACTTTAAATAAGACCAGTTATTTAAAGTTA
rox61
(SEQ ID NO: 19)
TAACTTTAAATAAGGCCCGTTATTTAAAGTTA
rox85
(SEQ ID NO: 20)
TAACTTTAAATAAGGCCGGTTATTTAAAGTTA

### VCre/VloxP

VloxP
(SEQ ID NO: 21)
TCAATTTCTGAGAACTGTCATTCTCGGAAATTGA

### SCre/SloxP

SloxP
(SEQ ID NO: 22)
CTCGTGTCCGATAACTGTAATTATCGGACATGAT

### Vika/vox

VOX
(SEQ ID NO: 24)
AATAGGTCTGAGAACGCCCATTCTCAGACGTATT

### FLP/frt

frt
(SEQ ID NO: 25)
GAAGTTCCTATACTTCTAGAAGAATAGGAACTTC

We claim:

1. A vector for gene expression comprising:
a promoter polynucleotide sequence;
a polynucleotide sequence encoding a first detectable protein in a first reading frame;
a polynucleotide sequence adapted for insertion of a test sequence;
a translation-inhibiting, nonsense-mediated decay polynucleotide sequence;
a start codon that is in a second reading frame;
a polynucleotide sequence encoding a second detectable protein in the first reading frame.

2. The vector for gene expression of claim 1, additionally comprising a polynucleotide sequence encoding a third detectable protein.

3. The vector for gene expression of claim 1, wherein the second reading frame is one reading frame removed from the first reading frame.

4. The vector for gene expression of claim 1, wherein the second reading frame is two reading frames removed from the first reading frame.

5. The vector for gene expression of claim 4, wherein the translation-inhibiting, nonsense-mediated decay polynucleotide sequence comprises at least one sequence selected from group consisting of a spacer sequence and an intron sequence.

6. The vector for gene expression of claim 3, wherein the translation-inhibiting, nonsense-mediated decay polynucleotide sequence comprises at least one sequence selected from group consisting of a spacer sequence and an intron sequence.

7. The vector for gene expression of claim 1, additionally comprising a polynucleotide sequence encoding a third detectable protein, flanked on the 5 and 3′ ends by recombination sequences.

8. The vector for gene expression of claim 7, wherein the recombination sequences are loxP sequences or rox sequences.

9. A method of inducing expression of a protein, the method comprising:
providing a vector comprising a promoter polynucleotide sequence, a polynucleotide sequence encoding a first detectable protein in a first reading frame, a test sequence encoding the protein to be expressed, a translation-inhibiting, nonsense-mediated decay polynucleotide sequence, a start codon that is in a second reading frame, and a polynucleotide sequence encoding a second detectable protein in the first reading frame; and
inducing expression from the promoter polynucleotide sequence.

10. The method of claim 9, wherein the vector additionally comprises a polynucleotide sequence encoding a third detectable protein.

11. The method of claim 9, wherein the second reading frame is one reading frame removed from the first reading frame.

12. The method of claim 9, wherein the second reading frame is two reading frames removed from the first reading frame.

13. The method of claim 12, wherein the translation-inhibiting, nonsense-mediated decay polynucleotide sequence comprises at least one sequence selected from group consisting of a spacer sequence and an intron sequence.

14. The method of claim 11, wherein the translation-inhibiting, nonsense-mediated decay polynucleotide sequence comprises at least one sequence selected from group consisting of a spacer sequence and an intron sequence.

15. The method of claim 9, wherein the vector additionally comprises a polynucleotide sequence encoding a third detectable protein, flanked on the 5 and 3′ ends by recombination sequences.

16. The method of claim 15, wherein the recombination sequences are loxP sequences or rox sequences.

17. A vector for gene expression comprising:
a promoter polynucleotide sequence;
a polynucleotide sequence encoding a first detectable protein;
first and second complementary recombination polynucleotide sequences flanking the polynucleotide sequence encoding a first detectable protein;
a polynucleotide sequence encoding a second detectable protein;
third and fourth complementary recombination polynucleotide sequences flanking the polynucleotide sequence encoding a second detectable protein;
a polynucleotide sequence encoding a third detectable protein.

18. The vector of claim 17, additionally comprising a nucleic acid sequence encoding a gene to be substituted into a host cell.

19. The vector of claim 18, wherein the complementary splice acceptor polynucleotide sequences are selected from the group consisting of loxP sequences and rox sequences.

20. The vector of claim 17, wherein the complementary splice acceptor polynucleotide sequences are selected from the group consisting of loxP sequences and rox sequences.
